SEQUENCE LISTING (4 sequences)

SEQ ID NO: 1 - DNA, 3348 bp, Mus musculus
gggcacgtgggatcggctgcagcactgcgccagttcagcccgcctagcagcgagcggccgcggcctctggagagaggttcctccccctctgctctgcaccagtccgagggaatctgtggtcagtgacgcgcatccttcagcgagccacctgcagcccggggcgcgccgctgggaccgcatcacaggctgggccggcggcctggctaccgcgcgctccggaggccatgcgggcgttggcgcagcgcagcgaccggcggctgctgctgcttgttgttttgtcagtaatgattcttgagaccgttacaaaccaagacctgcctgtgatcaagtgtgttttaatcagtcatgagaacaatggctcatcagcgggaaagccatcatcgtaccgaatgaggaatcgtttccatggccatcttgaacgtgacagagacccaggcaggagaatacctactccatattcagagcgaagccgccaactacacagtactgttcacagtgaatgtaagagatacacagctgtacgtgctaagaagaccttactttaggaagatggaaaaccaggacgcactgctctgcatctccgagggtgttccagagcccactgtggagtgggtgctctgcagctcccacagggaaagctgtaaagaagaaggccctgctgttgtcagaaaggaggaaaaggtacttcatgagttgttcggaacagacatcagatgctgtgctagaaatgcactgggccgcgaatgcaccaagctgttcaccatagatctaaaccaggctcctcagagcacactgccccagttattcctgaaagtgggggaacccttgtggatcaggtgtaaggccatccatgtgaaccatggattcgggctcacctgggagctggaagacaaagccctggaggagggcagctactttgagatgagtacctactccacaaacaggaccatgattcggattctcttggcctttgtgtcttccgtgggaaggaacgacaccggatattacacctgctcttcctcaaagcaccccagccagtcagcgttggtgaccatcctagaaaaagggtttataaacgctaccagctcgcaagaagagtatgaaattgacccgtacgaaaagttctgcttctcagtcaggtttaaagcgtacccacgaatccgatgcacgtggatcttctctcaagcctcatttccttgtgaacagagaggcctggaggatgggtacagcatatctaaattttgcgatcataagaacaagccaggagagtacatattctatgcagaaaatgatgacgcccagttcaccaaaatgttcacgctgaatataagaaagaaacctcaagtgctagcaaatgcctcagccagccaggcgtcctgttcctctgatggctacccgctaccctcttggacctggaagaagtgttcggacaaatctcccaattgcacggaggaaatcccagaaggagtttggaataaaaaggctaacagaaaagtgtttggccagtgggtgtcgagcagtactctaaatatgagtgaggccgggaaagggcttctggtcaaatgctgtgcgtacaattctatgggcacgtcttgcgaaaccatctttttaaactcaccaggccccttccctttcatccaagacaacatctccttctatgcgaccattgggctctgtctccccttcattgttgttctcattgtgttgatctgccacaaatacaaaaagcaatttaggtacgagagtcagctgcagatgatccaggtgactggccccctggataacgagtacttctacgttgacttcagggactatgaatatgaccttaagtgggagttcccgagagagaacttagagtttgggaaggtcctggggtctggcgctttcgggagggtgatgaacgccacggcctatggcattagtaaaacgggagtctcaattcaggtggcggtgaagatgctaaaagagaaagctgacagctgtgaaaaagaagctctcatgtcggagctcaaaatgatgacccacctgggacaccatgacaacatcgtgaatctgctgggggcatgcacactgtcagggccagtgtacttgatttttgaatattgttgctatggtgacctcctcaactacctaagaagtaaaagagagaagtttcacaggacatggacagagatttttaaggaacataatttcagtttttaccctactttccaggcacattcaaattccagcttcagaatgaattaaattcccattgaaccctgagagctgatccaagggcgggtgtaactgaacttctcgtgaaccaggcatgatgagattgaatatgaaaaccagaagaggctggcagaagaagaggaggaagatttgaacgtgctgacgtttgaagacctcctttgctttgcgtaccaagtggccaaaggcatggaattcctggagttcaagtcgtgtgtccacagagacctggcagccaggaatgtgttggtcacccacgggaaggtggtgaagatctgtgactttggactggcccgagacatcctgagcgactccagctacgtcgtcaggggcaacgcacggctgccggtgaagtggatggcacctgagagcttatttgaagggatctacacaatcaagagtgacgtctggtcctacggcatccttctctgggagatattttcactgggtgtgaacccttaccctggcattcctgtcgacgctaacttctataaactgattcagagtggatttaaaatggagcagccattctatgccacagaagggatatgtatcagaacatgggtggcaacgtcccagaacatccatccatctaccaaaacaggcggcccctcagcagagaggcaggctcagagccgccatcgccacaggcccaggtgaagattcacggagaaagaagttagcgaggaggccttggaccccgccaccctagcaggctgtagaccacagagccaagattagcctcgcctctgaggaagcgccctacaggccgttgcttcgctggacttttctctagatgctgtctgccattactccaaagtgacttctataaaatcaaacctctcctcgcacaggtgggagagccaataatgagacttgttggtgagcccgcctaccctggggggcctttccaggccccccaggcttgaggggaaagccatgtatctgaaatatagtatattcttgtaaatacgtgaaacaaaccaaacccgttttttgctaagggaaagctaaatatgatttttaaaaatctatgttttaaaatactatgtaactttttcatctatttagtgatatattttatggatggaaataaactttctactgtagaaa

SEQ ID NO: 2 - DNA, 20 bp, Artificial Sequence
  Synthetic
aagtgcagctcgccacccca

SEQ ID NO: 3 - DNA, 22 bp, Artificial Sequence
  Synthetic
ggtaccagcagagttggatagc

SEQ ID NO: 4 - DNA, 22 bp, Artificial Sequence
  Synthetic
atcccttacacagaagctggag

(19) United States
(12) Patent Application Publication
(10) Pub. No.: US 2023/0270085 A1
(43) Pub. Date: Aug. 31, 2023

(54) TRANSGENIC MOUSE MODEL EXPRESSING HUMAN HLA-A201 RESTRICTION GENE

(71) Applicant: The Jackson Laboratory, Bar Harbor, ME (US)

(72) Inventors: Anna Karolina Palucka, Avon, CT (US); Chu l. Yu, New Britain, CT (US); Jacques Banchereau, Farmington, CT (US); Richard Maser, Bar Harbor, ME (US); Leonard D. Shultz, Bar Harbor, ME (US)

(73) Assignee: The Jackson Laboratory, Bar Harbor, ME (US)

(21) Appl. No.: 18/015,043

(22) PCT Filed: Jul. 7, 2021
(86) PCT No.: PCT/US21/40682

§ 371 Date: Jan. 6, 2023

Related U.S. Application Data
(60) Provisional application No. 63/049,187, filed on Jul. 8, 2020.

(51) Int. Cl. A01K 67/027; C07K 14/54; C07K 14/535; C07K 14/74; C12N 15/85

(52) U.S. Cl. CPC A01K 67/0275; C07K 14/5403; C07K 14/535; C07K 14/70539; C12N 15/8509; A01K 2207/15; A01K 2217/075; A01K 2227/105; A01K 2267/0362; A01K 2267/0387; A01K 2217/072

(57) ABSTRACT

The present disclosure provides an immunodeficient NOD.Cg-Prkdc^{scid}Il2rg^{tm1Wjl}/SzJ (NSG™) mouse models that comprise an inactivated mouse Flt3 allele, a nucleic acid encoding human interleukin 3 (IL3), a nucleic acid encoding human granulocyte/macrophage-stimulating factor (GM-CSF), a nucleic acid encoding human stem cell factor (SCF), and a HLA-A2/H2-D/B2M transgene encoding (i) a human B2-microglubulin (B2M) covalently linked to MHC class 1, alpha 1, and alpha2 binding domains of a human HLA-A2.1 gene and (ii) alpha3 cytoplasmic and transmembrane domains of murine H2-db.

### RELATED APPLICATION

This application claims the benefit under 35 U.S.C. § 119(e) of U.S. provisional application No. 63/049,187, filed Jul. 8, 2020, which is incorporated by reference herein in its entirety.

### BACKGROUND

Mouse models have been used extensively to study human diseases in vivo to circumvent the complexity dealing with human patients. Nevertheless, murine models often inadequately recapitulate the human disease partly due to important differences between mouse and human immune systems (Hagai et al., 2018; Kanazawa, 2007; Mestas & Hughes, 2004; Williams, Flavell, & Eisenbarth, 2010). Thus, humanized mice, defined as mice with human immune system, could be an attractive alternative (Shultz, Brehm, Garcia-Martinez, & Greiner, 2012; Theocharides, Rongvaux, Fritsch, Flavell, & Manz, 2016; Victor Garcia, 2016; Zhang & Su, 2012). To this end immunodeficient mice lacking common gamma chain (γc) like NOD-SCID-Il12γc^{-/-} (NSG), or BALB/c-Rag2^{-/-}-γc^{-/-} (BRG) (Matsumura et al., 2003; Traggiai et al., 2004) can be humanized by transplantation of human CD34⁺ hematopoietic progenitor cells (HPCs). Based on the sources of T cells, the model can be further categorized into two types: (1) a model in which mature T cells are isolated from the donor of HPCs and adoptively transferred (Aspord et al., 2007; Pedroza-Gonzalez et al., 2011; Wu et al., 2014; Wu et al., 2018; Yu et al., 2008); in this case the T cells have been selected in human thymus; and (2) a model in which endogenous T cells are de novo generated from human CD34⁺ HPCs (Matsumura et al., 2003; Traggiai et al., 2004); in which case human T cells are selected in mouse thymus.

### SUMMARY

The present disclosure provides a humanized mouse model expressing the HLA-A201 restriction gene. One important aspect of humanized mouse research is the maturation of human adaptive immunity in the context of human MHC (Billerbeck et al., 2013; Danner et al., 2011; Najima et al., 2016). This mouse model was generated, in part, to support antigen presentation on human HLA and to match hematopoietic progenitor cell (HPC) donors with mice. This mouse model addresses, inter alia, limitations of the models discussed above. The biggest limitation of the first model in which mature T cells are isolated from the donor of HPCs and adoptively transferred is graft-versus-host disease; the biggest limitation of the second model in which endogenous T cells are de novo generated from human CD34⁺ HPCs is a limited number of T cells able to recognize human major histocompatibility complex (MHC).

Thus, some aspects of the present disclosure provide a non-obese diabetic (NOD) mouse comprising an inactivated mouse Prkdc allele, an inactivated mouse IL2rg allele, an inactivated mouse Flt3 allele, a nucleic acid encoding human interleukin 3 (IL3), a nucleic acid encoding human granulocyte/macrophage-stimulating factor (GM-CSF), a nucleic acid encoding human stem cell factor (SCF), and a nucleic acid encoding a human B2-microglubulin (B2M) covalently linked to MHC class 1, alpha1, and alpha2 binding domains of a human HLA-A2.1 gene, and alpha3 cytoplasmic and transmembrane domains of murine H2-db (HLA-A2/H2-D/B2M). Further aspects of the present disclosure provide an NSG™ mouse Comprising an inactivated mouse Flt3 allele; a nucleic acid encoding human IL3; a nucleic acid encoding human GM-CSF; a nucleic acid encoding human SCF; and a nucleic acid encoding HLA-A2/H2-D/B2M. These mouse models support antigen presentation on human HLA and allow matching of hematopoietic progenitor cell (HPC) donors with mice.

Also provided herein are methods of producing an NOD mouse comprising an inactivated mouse Prkdc allele, an inactivated mouse IL2rg allele, an inactivated mouse Flt3 allele, a nucleic acid encoding human IL3, a nucleic acid encoding human GM-CSF, a nucleic acid encoding human SCF, and a nucleic acid encoding a human HLA-A2/H2-D/B2M, methods of using the mouse as a model system, and methods of propagating the mouse.

Further provided herein are NSG™ cells comprising a nucleic acid encoding human ILS, a nucleic acid encoding human GM-CSF, a nucleic acid encoding human SCF, and a transgene encoding human HLA-A2/H2-D/B2M.

### BRIEF DESCRIPTION OF THE DRAWINGS

FIGS. 1A-1C depict human engraftment from different sources of human CD34⁺ HPCs in NSG-SGM3F-A2 mice. FIG. 1A is a schematic depicting the breeding scheme of NSG-SGM3F-A2 mice. FIG. 1B depicts HLA-A2 expression on mCD45⁺ cells in the blood of 4 weeks-old mice. FIG. 1C depicts graphs measuring the absolute number of hCD45⁺ cells and the percentage of human CD33⁺, CD19⁺, and CD3⁺ cells in hNSG-SGM3F-A2 at 12 weeks post-transplant with human fetal liver, cord blood, and bone marrow HPCs.

FIGS. 2A-2D depict the comparison of human engraftment in humanized SGM3F-A2 mice engrafted with human cord blood or fetal liver HPCs. FIG. 2A depicts human engraftment measured in the blood by the percentage and the absolute number of hCD45⁺ cells in hSGM3F-A2 mice at 12 weeks post-transplant with 1×10⁵ cord blood (CB) or fetal liver (FL) HPCs. n=91 mice from 5 CB donors, n=95 mice from 4 FL donors. Nested t test. FIG. 2B depicts the absolute number of hCD33⁺, hCD19⁺, hCD3⁺ cells in hSGM3F-A2 mice. FIG. 2C depicts the absolute number of human CD4⁺ T cells and CD8⁺ T cells in the blood of hSGM3F-A2 mice. FIG. 2D depicts total human IgM, IgG and IgA measured in the plasma of hSGM3F-A2 mice at 12-week post-transplant by ELISA.

### DETAILED DESCRIPTION

The present disclosure provides mouse models that support antigen presentation on human HLA and matches hematopoietic progenitor cell (HPC) donors with mice. The mouse models provide herein, in some aspects, have a NOD.Cg-Prkdcscid Il2rgtm1Wjl/SzJ (NSG™) background and further comprise an inactivated mouse Flt3 allele, a nucleic acid encoding human interleukin 3 (IL3), a nucleic acid encoding human granulocyte macrophage-colony stimulating factor (GM-CSF), a nucleic acid encoding human stem cell factor (SCF), and a nucleic acid encoding a human B2-microglubulin (B2M) covalently linked to the MHC class 1, alpha1 and alpha2 binding domains of the human HLA-A2.1 gene, and the alpha3, cytoplasmic and transmembrane domains of the murine H2-db (referred to herein as NSG-SGM3F-A2 mice). In some embodiments, the genotype of an NSG-SGM3F-A2 mouse model is NOD.Cg-Prkdc^{scid} Il2rg^{tm1Wjl} Tg(HLA-A/H2-D/B2M)^{1Dvs/SzJ} Flt3^{em1Akp} Tg(CMV-IL3,CSF2,KITLG)^{1Eav/MloySzJ} (see Example 1 for an exemplary method of generating the NOD.Cg-Prkdc^{scid} Il2rg^{tm1Wjl} Tg(HLA-A/H2-D/B2M)^{1Dvs/SzJ} Flt3^{em1Akp} Tg(CMV-IL3,CSF2,KITLG)^{1Eav/MloySzJ} mouse). In some embodiments, NOD.Cg-Prkdc^{scid} Il2rg^{tm1Wjl}-Flt3^{em1Akp} Tg(CMV-IL3,CSF2,KITLG)^{1Eav/MloySzJ} (SGM3F) mice are crossed with HLA-A0201 transgenic mice (NSG-A2 (HHD)) and interbred until all offspring are homozygous to yield NOD.Cg-Prkdc^{scid} Il2rg^{tm1Wjl} Tg(HLA-A/H2-D/B2M)^{1Dvs/SzJ} Flt3^{em1Akp} Tg(CMV-IL3,CSF2,KITLG)^{1Eav/MloySzJ} mice.

The NSG™ mouse is an immunodeficient mouse that lacks mature T cells, B cells, and natural killer (NK) cells, is deficient in multiple cytokine signaling pathways, and has many defects in innate immunity (see, e.g., (Shultz, Ishikawa, & Greiner, 2007; Shultz et al., 2005; Shultz et al., 1995), each of which is incorporated herein by reference). The NSG™ mouse, derived from the non-obese diabetic (NOD) mouse strain NOD/ShiLtJ (see, e.g., (Makino et al., 1980), which is incorporated herein by reference), include the Prkdc^{scid} mutation (also referred to as the “severe combined immunodeficiency” mutation or the “scid” mutation) and the Il2rg^{tm1Wjl} targeted mutation. The Prkdc^{scid} mutation is a loss-of-function mutation in the mouse homolog of the human PRKDC gene—this mutation essentially eliminates adaptive immunity (see, e.g., (Blunt et al., 1995; Greiner, Hesselton, & Shultz, 1998), each of which is incorporated herein by reference). The Il2rg^{tm1Wjl} mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells ((Cao et al., 1995; Greiner et al., 1998; Shultz et al., 2005), each of which is incorporated herein by reference). A loss-of-function mutation, as is known in the art, results in a gene product with little or no function. By comparison, a null mutation results in a gene product with no function. An inactivated allele may be a loss-of-function allele or a null allele.

An inactivated allele is an allele that does not produce a detectable level of a functional gene product (e.g., a functional protein). In some embodiments, an inactivated allele is not transcribed. In some embodiments, an inactivated allele does not encode a functional protein. Thus, a mouse comprising an inactivated mouse Flt3 allele does not produce a detectable level of functional FLT3. In some embodiments, a mouse comprising an inactivated mouse Flt3 allele does not produce any functional FLT3.

Flt3 is a receptor important for development of the dendritic cells and monocytic lineages. Flt3L-Flt3 signaling is important for the development of various DC and monocytic lineages (Ding et al., 2014; Ginhoux et al., 2009; McKenna et al., 2000; Waskow et al., 2008) and its role is further supported by the increase of circulating conventional (c)DCs and plasmacytoid (p)DCs after the administration of Flt3L in vivo in mice and humans (Karsunky, Merad, Cozzio, Weissman, & Manz, 2003; Maraskovsky et al., 1996; Pulendran et al., 2000). Knocking-out mouse Flt3 can lead to: (1) decrease in murine DCs and other myeloid cells; and (2) increase in the availability of mouse Flt3L (which can act via human receptor) to human cells, thereby improving the long-term development of human myeloid cells upon transplant with human CD34⁺ HPCs.

The NSG-SGM3F-A2 mouse models provide herein comprise a genomic modification that inactivates the mouse Flt3 allele. A modification, with respect to a nucleic acid, is any manipulation of the nucleic acid, relative to the corresponding wild-type nucleic acid (e.g., the naturally-occurring nucleic acid). A genomic modification is thus any manipulation of a nucleic acid in a genome, relative to the corresponding wild-type nucleic acid (e.g., the naturally-occurring nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g. protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs). In some embodiments, CRISPR/Cas gene editing is used to inactivate the mouse Flt3 allele, as described elsewhere herein.

In some embodiments, a genomic modification (e.g., a deletion or an indel) is in a (at least one) region of the mouse Flt3 allele selected from coding regions, non-coding regions, and regulatory regions. In some embodiments, the genomic modification (e.g., a deletion or an indel) is a coding region of the mouse Flt3 allele. For example, the genomic modification (e.g., a deletion or an indel) may be in exon 3, or it may span exon 3 of the mouse Flt3 allele. In some embodiments, the genomic modification is a genomic deletion. For example, the mouse Flt3 allele may comprise a genomic deletion of nucleotide sequences in exon 3. In some embodiments, the nucleotide sequence of SEQ ID NO: 1 has been deleted from an inactivated mouse Flt3 allele. In some embodiments, an inactivated mouse Flt3 allele comprises the nucleotide sequence of SEQ ID NO: 1.

In some embodiments, the NSG-SGM3F-A2 mouse models provided herein do not express a detectable level of mouse FLT3. A detectable level of mouse FLT3 is any level of FLT3 protein detected using a standard protein detection assay, such as flow cytometry and/or an ELISA. In some embodiments, an NSG-SGM3F-A2 mouse model expresses an undetectable level or a low level of mouse FLT3. For example, a mouse model may express less than 1,000 pg/ml mouse FLT3. In some embodiments, mouse model expresses less than 500 pg/ml mouse FLT3 or less than 100 pg/ml mouse FLT3. The mouse FLT3 receptor is also referred to as cluster of differentiation antigen CD135. Thus, in some embodiments, an NSG-SGM3F-A2 mouse model does not comprise (there is an absence of) CD135⁺ multipotent progenitor cells.

Flt3 knockout mice, in some embodiments, are generated by CRISPR using Cas9 mRNA and a guide RNA (gRNA). In some embodiments, the gRNA (e.g., 5′-AAGTGCAGCTCGCCACCCCA-3′, SEQ ID NO: 2) targets exon 3 of mouse Flt3 NSG™ mice (NOD.Cg-Prkdc^{scid} Il2rg^{tm1Wjl}-Flt3^{em1Akp}; RRID:IMSR JAX:005557). The blastocysts derived from the injected embryos, in some embodiments, are transplanted into foster mothers and newborn pups are obtained. In some embodiments, mice carrying a null deletion are backcrossed to NSG™. F0 and F1 littermates may be tested for successful gene-knockout by PCR and Sanger sequencing, for example. For example, primers (5′-GGTACCAGCAGAGTTGGATAGC-3′, SEQ ID NO: 3) and (5′-ATCCCTTACACAGAAGCTGGAG-3′, SEQ ID NO: 4) may be used in a PCR reaction to detect the mouse Flt3 wildtype allele from mutant allele (Table 1). The WT allele yields a DNA fragment 799 bp in length, whereas the mutated allele generates a DNA fragment of 363 bp in length.

### Transgenic Mouse Models

Transgenic mouse models (Tg mice) can be generated to modify a gene sequence, for example, by substituting the gene sequence with a transgene, or by adding a gene sequence that is not found within the locus. The NSG-SGM3F-A2 mouse models provided herein include a transgenic allele. They include an exogenous nucleic acid that has been introduced into the mouse genome.

A nucleic acid used as provided herein may be a DNA, an RNA, or a chimera of DNA and RNA. In some embodiments, a nucleic acid (e.g., DNA) comprises a gene encoding a particular protein of interest. A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences). As is known in the art, a promoter sequence is a DNA sequence at which transcription of a gene begins. Promoter sequences are typically located directly upstream of (at the 5′ end of) a transcription initiation site. An exon is a region of a gene that codes for amino acids. An intron (and other non-coding DNA) is a region of a gene that does not code for amino acids.

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene).

Methods of producing a transgenic mouse model are described elsewhere herein.

The NSG-SGM3F-A2 mice described herein comprise an inactivated mouse Flt3 allele, a nucleic acid encoding IL3, a nucleic acid encoding GM-CSF, a nucleic acid encoding SCF, and a nucleic acid encoding a human B2-microglubulin (B2M) covalently linked to the MHC class 1, alpha1 and alpha2 binding domains of the human HLA-A2.1 gene, and the alpha3, cytoplasmic and transmembrane domains of the murine H2-db. In some embodiments, the NSG-SGM3F-A2 mice described herein comprise an inactivated mouse Flt3 allele, a nucleic acid encoding human IL3, a nucleic acid encoding human GM-CSF, a nucleic acid encoding human SCF, and a nucleic acid encoding human B2-microglubulin (B2M) covalently linked to the MHC class 1, alpha1 and alpha2 binding domains of the human HLA-A2.1 gene, and the alpha3, cytoplasmic and transmembrane domains of the murine H2-db. In some embodiments, the NSG-SGM3F-A2 mice comprise a human IL3 transgene, a human GM-CSF transgene, a human SCF transgene, and a human HLA-A2/H2-D/B2M transgene (a transgene encoding a human B2-microglubulin (B2M) covalently linked to the MHC class 1, alpha1 and alpha2 binding domains of the human HLA-A2.1 gene, and the alpha3, cytoplasmic and transmembrane domains of the murine H2-db). In some embodiments, a transgene, such as a human IL3 transgene, a human GM-CSF transgene, a human SCF transgene, and/or a human HLA-A2/H2-D/B2M transgene, is integrated into a mouse genome. Human IL3, CSF2, and KITLG transgenes are described (Nicolini, Cashman, Hogge, Humphries, & Eaves, 2004), incorporated by reference herein. Human HLA-A2/H2-D/B2M transgene is described (Pascolo et al., 1997; Takaki et al., 2006), incorporated by reference herein.

NSG-SGM3F-A2 mice, in some embodiments, are generated by crossing NSG-HLA-A2/HHD mice (RRID:IMSR JAX: 014570) to SGM3F mice (NOD.Cg-Prkdc^{scid} Il2rg^{tm1Wjl}-Flt3^{em1Akp} Tg(CMV-IL3,CSF2,KITLG)^{1Eav/MloySzJ}). NSG-SGM3 mice carry three separate transgenes which were designed each carrying one of the human interleukin-3 (IL3) gene, the human granulocyte/macrophage-stimulating factor (GM-CSF) gene, or human stem cell factor (SCF) gene. Expression of each gene is driven by a human cytomegalovirus promoter/enhancer sequence and is followed by a human growth hormone cassette and a polyadenylation (polyA) sequence. The transgenes were microinjected into fertilized C57BL/6xC3H/HeN oocytes. The resulting founders, carrying all three transgenes (3GS), in some embodiments, are backcrossed to BALB/c-scid/scid mice for several generations and subsequently backcrossed to NOD.CB17-Prkdcscid mice for multiple (e.g., at least 11) generations (Nicolini et al., 2004; Wunderlich et al., 2010). These mice may then be bred to NSG mice (NOD.Cg-Prkdc^{scid} Il2rg^{tm1Wjl}; RRID:IMSR JAX: 005557), for example, and then interbred until all offspring are homozygous for 3GS and the IL2rg targeted mutation. The transgenic mice may be bred to NSG mice for at least one generation to establish NSG-SGM3 mice. NSGF mice may be generated, for example, using the CRISPR/cas system. Cas9 mRNA and sgRNAs targeting mouse Flt3, in some embodiments, are coinjected into fertilized NSG oocytes. The resulting founders, carrying Flt3 deletion may be bred to NSG mice, and then interbred until all offspring are homozygous for Flt3 targeted mutation. The NSG-SGM3 mice may be bred to NSGF mice for multiple (e.g., two) generations to establish NSG-SGM3F mice. HLA-A2/HHD transgenic expression in H-2Db^{-/-} B2m^{-/-} mice restore CD8+ T cells and enable HLA-A2.1-restricted cytotoxic T cell response (Pascolo et al., 1997). NSG-HLA-A2/HHD mice may then be bred to NSG-SGM3F mice for multiple (e.g., at least four) generations to establish NSG-SGM3F-A2 mice.

### Human Immune System Model

The NSG-SGM3F-A2 mouse models of the present disclosure, in some embodiments, are used to support human CD34⁺ hematopoietic progenitor cells (HPCs) and development of a human innate immune system. The human immune system includes the innate immune system and the adaptive immune system. The innate immune system is responsible for recruiting immune cells to sites of infection, activation of the complement cascade, the identification and removal of foreign substances from the body by leukocytes, activation of the adaptive immune system, and acting as a physical and chemical barrier to infectious agents.

In some embodiments, an NSG-SGM3F-A2 mouse model provided herein is sublethally irradiated (e.g., 100-300 cGy) to kill resident mouse HPCs, and then the irradiated mouse is engrafted with human CD34⁺ HPCs (e.g., 50,000 to 200,000 HPCs) to initiate the development of a human innate immune system. Thus, in some embodiments, a mouse further comprises human CD34⁺ HPCs. Human CD34⁺ HPCs may be from any source including, but not limited to, human fetal liver, umbilical cord blood, mobilized peripheral blood, and bone marrow. In some embodiments, human CD34⁺ HPCs are from human umbilical cord blood.

The differentiation of human CD34⁺ HPCs into divergent immune cells (e.g., T cells, B cells, dendritic cells) is a complex process in which successive developmental steps are regulated by multiple cytokines. This process can be monitored through cell surface antigens, such as cluster of differentiation (CD) antigens. CD45, for example, is expressed on the surface of HPCs, macrophages, monocytes, T cells, B cells, natural killer cells, and dendritic cells, thus can be used as a marker indicative of engraftment. On T cells, CD45 regulates T cell receptor signaling, cell growth, and cell differentiation. In some embodiments, an NSG-SGM3F-A2 mouse model comprises human CD45⁺ cells. In some embodiments, an NSG-SGM3F-A2 mouse model also exhibits engraftment of human CD45⁺ cells to tissues, but not limited to, in the lung, thymus, spleen, lymph nodes, and/or small intestine.

As CD45⁺ cells mature, they begin to express additional biomarkers, indicative of the various developmental stages and differentiating cell types. Developing T cells, for example, also express CD3, CD4, and CD8. As another example, developing myeloid cells express CD33⁺. An mouse model herein, in some embodiments, comprises not only human CD45⁺ cells but also double positive human CD45⁺/CD3⁺ T cells as well as double positive human CD45⁺/CD33⁺ myeloid cells.

Thus, in some embodiments, a population of human CD45⁺ cells in an NSG-SGM3F-A2 mouse model comprises human CD45⁺/CD3⁺ T cells. In some embodiments, the population of human CD45⁺ cells comprise an increased percentage of human CD45⁺/CD3⁺ T cells, relative to an NSG™ control mouse. In some embodiments, the percentage of human CD45⁺/CD3⁺ T cells in an NSG-SGM3F-A2 mouse model is increased by at least 25%, relative to an NSG™ control mouse. For example, the percentage of human CD45⁺/CD3⁺ T cells in a mouse model may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse. In some embodiments, the percentage of human CD45⁺/CD3⁺ T cells in a mouse model is increased by at least 50%, relative to an NSG™ control mouse. In some embodiments, the percentage of human CD45⁺/CD3⁺ T cells in a mouse model is increased by at least 100%, relative to an NSG™ control mouse. In some embodiments, the percentage of human CD45⁺/CD3⁺ T cells in a mouse model is increased by 25%-100%, 25%-75%, 25%-50%, 50%-100%, 50%-75%, or 75%-100%, relative to an NSG™ control mouse.

In some embodiments, a population of human CD45⁺ cells in an NSG-SGM3F-A2 mouse model comprises human CD45⁺/CD33⁺ myeloid cells. In some embodiments, the population of human CD45⁺ cells comprise an increased percentage of human CD45⁺/CD33⁺ myeloid cells, relative to an NSG™ control mouse. In some embodiments, the percentage of human CD45⁺/CD33⁺ T cells in a mouse model is increased by at least 25%, relative to an NSG™ control mouse. For example, the percentage of human CD45⁺/CD33⁺ myeloid cells in a mouse model may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse. In some embodiments, the percentage of human CD45⁺/CD33⁺ myeloid cells in a mouse model is increased by at least 50%, relative to an NSG™ control mouse. In some embodiments, the percentage of human CD45⁺/CD33⁺ myeloid cells in a mouse model is increased by at least 100%, relative to an NSG™ control mouse. In some embodiments, the percentage of human CD45⁺/CD33⁺ myeloid cells in a mouse model is increased by 25%-100%, 25%-75%, 25%-50%, 50%-100%, 50%-75%, or 75%-100%, relative to an NSG™ control mouse.

In some embodiments, a population of human CD45⁺ cells in an NSG-SGM3F-A2 mouse model comprises human CD45⁺/CD19⁺ B cells. In some embodiments, the population of human CD45⁺ cells comprise an increased percentage of human CD45⁺/CD19⁺ B cells, relative to an NSG™ control mouse. In some embodiments, the percentage of human CD45⁺/CD19⁺ B cells in a mouse model is increased by at least 25%, relative to an NSG™ control mouse. For example, the percentage of human CD45⁺/CD19⁺ B cells in a mouse model may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse. In some embodiments, the percentage of human CD45⁺/CD19⁺ B cells in a mouse model is increased by at least 50%, relative to an NSG™ control mouse. In some embodiments, the percentage of human CD45⁺/CD19⁺ B cells in a mouse model is increased by at least 100%, relative to an NSG™ control mouse. In some embodiments, the percentage of human CD45⁺/CD19⁺ B cells in a mouse model is increased by 25%-100%, 25%-75%, 25%-50%, 50%-100%, 50%-75%, or 75%-100%, relative to an NSG™ control mouse.

The NSG-SGM3F-A2 mouse models provided herein, surprisingly, are also capable of supporting engraftment of dendritic cells (e.g., plasmacytoid dendritic cells and myeloid dendritic cells), natural killer cells, and monocyte-derived macrophages (monocyte macrophages). Plasmacytoid dendritic cells (pDCs) secrete high levels of interferon alpha; myeloid dendritic cells (mDCs) secrete interleukin 12, interleukin 6, tumor necrosis factor, and chemokines; natural killer cells destroy damaged host cells, such as tumor cells and virus-infected cells; and macrophages consume substantial numbers of bacteria or other cells or microbes.

In some embodiments, an NSG-SGM3F-A2 mouse model comprises an increased percentage of human CD11c⁺ myeloid dendritic cells, relative to an NSG™ control mouse and/or an NSGF control mouse. In some embodiments, the percentage of human CD11c⁺ HLA-DR⁺ myeloid dendritic cells in the NSG-SGM3F-A2 mouse is increased by at least 25%, relative to an NSG™ control mouse and/or an NSGF control mouse. For example, the percentage of human CD11C⁺ HLA-DR⁺ myeloid dendritic cells in the NSG-SGM3F-A2 mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse and/or an NSGF control mouse. In some embodiments, the percentage of human CD11c⁺ HLA-DR⁺ myeloid dendritic cells in the NSG-SGM3F-A2 mouse is increased by at least 50%, relative to an NSG™ control mouse and/or an NSGF control mouse. In some embodiments, the percentage of human CD11c⁺ HLA-DR⁺ myeloid dendritic cells in the NSG-SGM3F-A2 mouse is increased by at least 100%, relative to an NSG™ control mouse and/or an NSGF control mouse. In some embodiments, the percentage of human CD11c⁺ HLA-DR⁺ myeloid dendritic cells in the NSG-SGM3F-A2 mouse is increased by 25%-100%, 25%-75%, 25%-50%, 50%-100%, 50%-75%, or 75%-100%, relative to an NSG™ control mouse and/or an NSGF control mouse.

In some embodiments, an NSG-SGM3F-A2 mouse model of the present disclosure is used to support engraftment of HLA-A2 matched hematopoietic lineages.

### Methods of Producing Transgenic Animals

Provided herein, in some aspects, are methods of producing a transgenic animal that expresses human transgenes. A transgenic animal, herein, refers to an animal that has a foreign (exogenous) nucleic acid (e.g., transgene) inserted into (integrated into) its genome. In some embodiments, the transgenic animal is a transgenic rodent, such as a mouse or a rat. In some embodiments, the transgenic animal is a mouse. Three conventional methods used for the production of transgenic animals include DNA microinjection (Gordon & Ruddle, 1981), incorporated herein by reference), embryonic stem cell-mediated gene transfer (Gossler, Doetschman, Korn, Serfling, & Kemler, 1986), incorporated herein by reference) and retrovirus-mediated gene transfer (Jaenisch, 1976), incorporated herein by reference), any of which may be used as provided herein. Electroporation may also be used to produce transgenic mice (see, e.g., WO 2016/054032 and WO 2017/124086, each of which is incorporated herein by reference).

A nucleic acid, in some embodiments, comprises a transgene, for example, a transgene that comprises a promoter (e.g., a constitutively active promoter) operably linked to a nucleotide sequence encoding a polypeptide of interest. In some embodiments, a nucleic acid used to produce a transgenic animal (e.g., mouse) is present on a vector, such as a plasmid, a bacterial artificial chromosome (BAC), or a yeast artificial chromosome (YAC), which is delivered, for example, to the pronucleus/nucleus of a fertilized embryo where the nucleic acid randomly integrates into the animal genome. In some embodiments, the fertilized embryo is a single-cell embryo (e.g., a zygote). In some embodiments, the fertilized embryo is a multi-cell embryo (e.g., a developmental stage following a zygote, such as a blastocyst). In some embodiments, a nucleic acid (e.g., carried on a BAC) is delivered to a fertilized embryo of mouse to produce a mouse model of the present disclosure. Following injection of the fertilized embryo, the fertilized embryo may be transferred to a pseudopregnant female, which subsequently gives birth to offspring comprising the nucleic acid encoding the polypeptide of interest. The presence or absence of the nucleic acid may be confirmed, for example, using any number of genotyping methods (e.g., sequencing and/or genomic PCR).

Also provided herein are methods of inactivating an endogenous Flt3 allele. In some embodiments, an endogenous Flt3 allele is inactivated in a transgenic animal. In some embodiments, a gene/genome editing method is used for gene (allele) inactivation. Engineered nuclease-based gene editing systems that may be used as provided herein include, for example, clustered regularly interspaced short palindromic repeat (CRISPR) systems, zinc-finger nucleases (ZFNs), and transcription activator-like effector nucleases (TALENs). See, e.g., (Carroll, 2011; Gaj, Gersbach, & Barbas, 2013; Joung & Sander, 2013), each of which is incorporated by reference herein.

In some embodiments, a CRISPR system is used to inactivate an endogenous Flt3 allele of an NSG-SGM3F-A2 mouse model provided herein. See, e.g., (Harms et al., 2014; Inui et al., 2014), each of which are incorporated by reference herein). For example, Cas9 mRNA or protein and one or multiple guide RNAs (gRNAs) can be injected directly into mouse embryos to generate precise genomic edits into a Flt3 gene. Mice that develop from these embryos can be genotyped or sequenced to determine if they carry the desired mutation(s), and those that do may be bred to confirm germline transmission.

The CRISPR/Cas system is a naturally occurring defense mechanism in prokaryotes that has been repurposed as a RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target to be modified. Thus, one can change the genomic target of the Cas protein by simply changing the target sequence present in the gRNA. In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence (e.g., a region of the Flt3 allele). See, e.g., (Deltcheva et al., 2011; Jinek et al., 2012), each of which is incorporated by reference herein. In some embodiments, a gRNA used in the methods provided herein binds to a region (e.g., exon 3) of a mouse Flt3 allele. In some embodiments, the gRNA that binds to a region of a mouse Flt3 allele comprises the nucleotide sequence of 5′-AAGTGCAGCTCGCCACCCCA-3′(SEQ ID NO: 2).

### METHODS OF USE

The NSG-SGM3F-A2 mouse models provided herein may be used for any number of applications. For example, a mouse model may be used to test how a particular agent (e.g., therapeutic agent) or medical procedure (e.g., tissue transplantation) impacts the human innate immune system (e.g., human innate immune cell responses) and human adaptive immune system (e.g., antibody response).

In some embodiments, a mouse model is used to evaluate an effect of an agent on human innate immune system development. Thus, provided herein are methods that comprise administering an agent to a mouse model, and evaluating an effect of the agent on human innate immune system development in the mouse. Effects of an agent may be evaluated, for example, by measuring a human innate immune cell (e.g., T cell and/or dendritic cell) response (e.g., cell death, cell signaling, cell proliferation, etc.) and human adaptive immune response (e.g., antibody production). Non-limiting examples of agents include therapeutic agents, such as anti-cancer agents and anti-inflammatory agents, and prophylactic agents, such as immunogenic compositions (e.g., vaccines).

In other embodiments, a mouse model is used to evaluate an immunotherapeutic response to a human tumor. Thus, provided herein are methods that comprise administering an agent to a mouse model that has a human tumor, and evaluating an effect of the agent on the human innate immune system and/or on the tumor in the mouse. Effects of an agent may be evaluated by measuring a human innate immune cell (e.g., T cell and/or dendritic cell) response, human adaptive immune response (e.g., antibody production) and/or tumor cell response (e.g., cell death, cell signaling, cell proliferation, etc.). In some embodiments, the agent is an anti-cancer agent.

In yet other embodiments, a mouse model is used to evaluate a human innate immune response to an infectious microorganism. Thus, provided herein are methods that comprise exposing a mouse model to an infectious microorganism (e.g., bacteria and/or virus), and evaluating an effect of the infectious microorganism on the human innate immune response.

Effects of an infectious microorganism may be evaluated by measuring a human innate immune cell (e.g., T cell and/or dendritic cell) response (e.g., cell death, cell signaling, cell proliferation, etc.). These methods may further comprise administering a drug or an anti-microbial agent (e.g., an anti-bacterial agent or an anti-viral agent) to the mouse, and evaluating an effect of the drug or anti-microbial agent on the infectious microorganism.

In still further embodiments, a mouse model is used to evaluate a human immune response to tissue transplantation. Thus, provided herein are methods that comprise transplanting tissue (e.g., allogeneic tissue) to a mouse model and evaluating an effect of the transplanted tissue on the human innate immune response. Effects of a transplanted tissue may be evaluated by measuring a human innate immune cell (e.g., T cell and/or dendritic cell) response (e.g., cell death, cell signaling, cell proliferation, etc.) and human adaptive immune response (e.g., antibody production) to the transplanted tissue.

### EXAMPLES

### Example 1. The NOD.Cg-Prkd^{scid} Il2rg^{tm1Wjl} Tg(HLA-A/H2-D/B2M)^{1Dvs/SzJ} Flt3^{em1Akp} Tg(CMV-IL3,CSF2,KITLG)^{1Eav/MloySzJ} (NSG-SGM3F-A2) Mouse Model

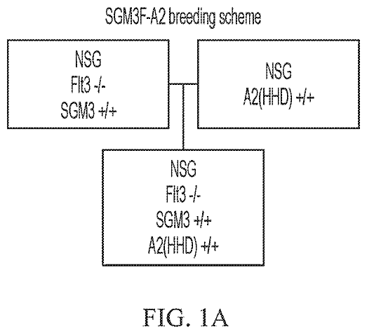

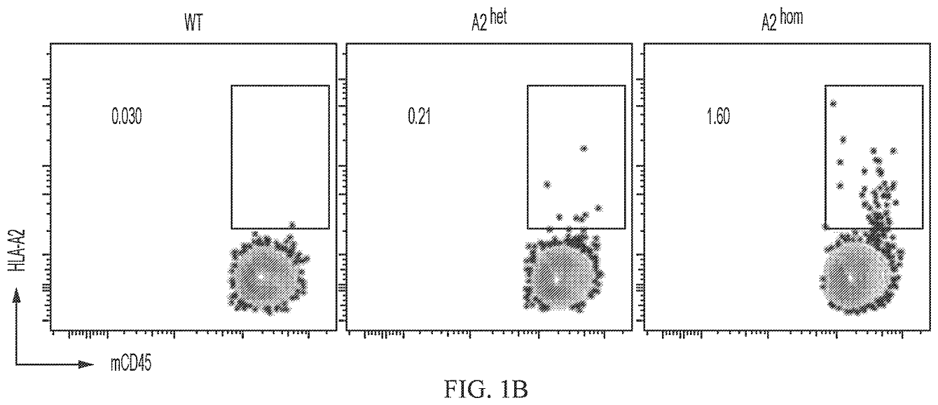

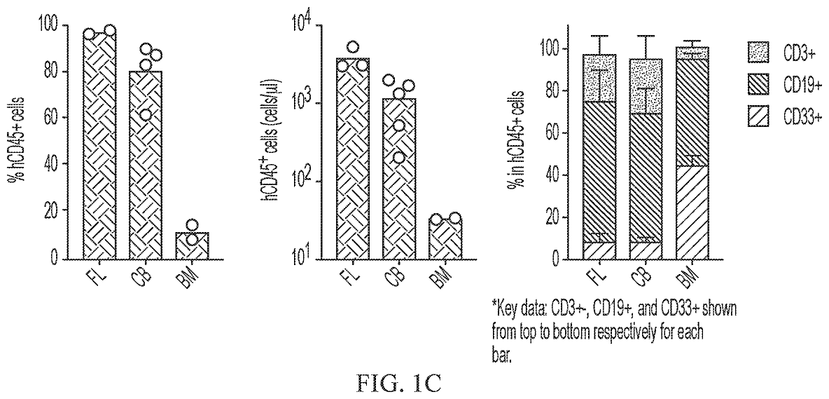

One important aspect of humanized mouse research is the maturation of human adaptive immunity in the context of human MHC (Billerbeck et al., 2013; Danner et al., 2011; Najima et al., 2016). To support antigen presentation on human HLA and to match HPC donors with mice, we crossed SGM3F (NOD.Cg-Prkdc^{scid} Il2rg^{tm1Wjl}-Flt3^{em1AkP} Tg(CMV-IL3,CSF2,KITLG)^{1Eav/MloySzJ}) mice with HLA-A0201 transgenic mice (NSG-A2 (HHD)) and interbred until all offspring were homozygous to yield NOD.Cg-Prkdc^{scid} Il2rg^{tm1Wjl} Tg(HLA-A/H2-D/B2M)^{1Dvs/SzJ} Flt3^{em1Akp} Tg(CMV-IL3,CSF2,KITLG)^{1Eav/MloySzJ} (NSG-SGM3F-A2) (FIG. 1A). SGM3F mice combined the features of NSG mice with transgenic expression of human stem cell factor (SCF), Granulocyte Macrophage-Colony Stimulating Factor (GM-CSF) and Interleukin (IL)-3 (NSG-SGM3, SGM3) (Nicolini et al., 2004; Wunderlich et al., 2010) and NSG mice with Flt3 mutant mice (NSGF). To confirm the expression of human HLA-A0201, we measured and confirmed the surface expression of HLA-A2 in mouse bone marrow cells (FIG. 1B). To test their capacity to support the engraftment of the human immune system, NSG-SGM3F-A2 mice were irradiated sub-lethally and transplanted with 1×10⁵ HLA-A2⁺ CD34⁺ HPCs from human fetal liver, cord blood or adult bone marrow. Mice that received both fetal liver and cord blood HPCs showed comparable immune cell composition in the blood while less CD3⁺ T cells were found in mice with bone marrow HPCs (FIG. 1C). Furthermore, at 6 months post-transplant with FL, CB, or BM HPCs, we observed high amount of hCD45⁺ immune cells in the lungs with different human immune cells that are important for both innate and adaptive immune response, including CD11c⁺ DCs and CD3⁺ T cells (data not shown). Importantly, at 6 months post-transplant with HLA-A2⁺ CB HPCs, HLA-A2 expression in thymus was detected on mouse thymic epithelial cells of hNSG-SGM3F-A2 mice reconstituted with HLA-A2⁺ HPCs using BB7.2 antibody specific to HLA-A2 (data not shown), which allow the maturation of T cells in the context of human HLA-A2.

### Example 2. The Comparison of Human Engraftment in Humanized SGM3F-A2 Mice Engrafted with Human Cord Blood or Fetal Liver HPCs

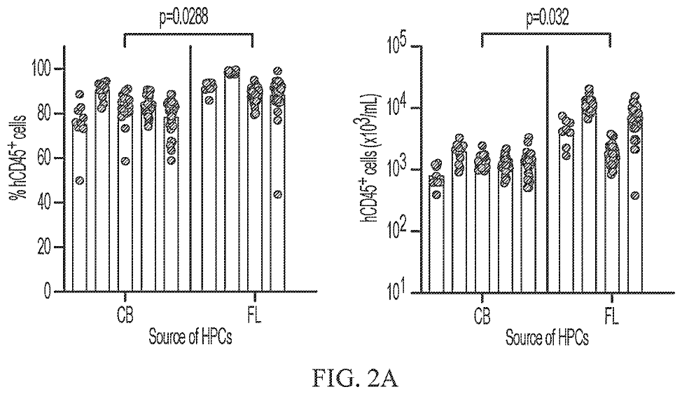

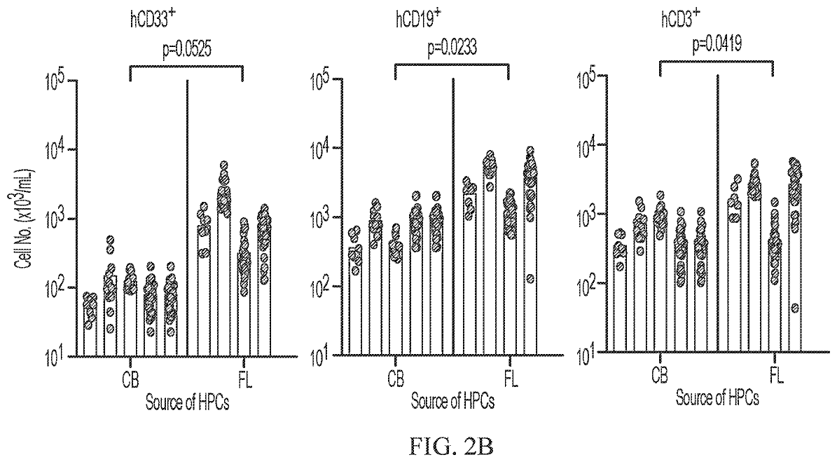

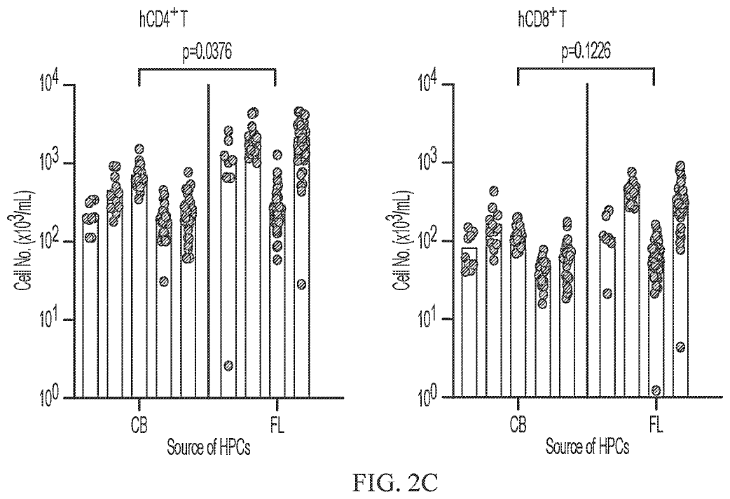

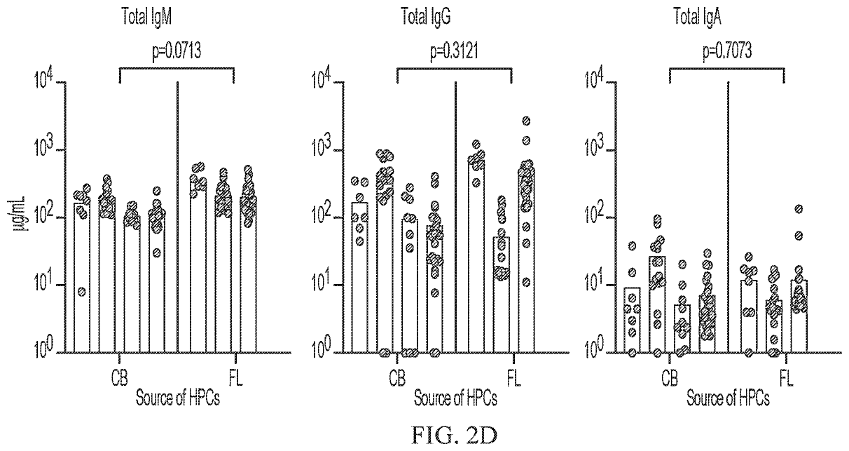

Due to limited availability of human fetal tissues, the use of cord blood derived HPCs to construct humanized mice was validated. A side-by-side comparison was performed on different cell types from cohorts of mice engrafted with 4-5 different cord blood or fetal liver donors. The data shows that humanized mice engrafted with fetal liver HPCs demonstrated only a slightly higher hCD45⁺ engraftment at 12-weeks post-transplant due to the expansion of hCD19+ B cells and hCD3+ T cells (FIGS. 2A-2B). In mice engrafted with fetal liver HPCs, a slight increase in hCD4⁺ T cells was observed, but no difference was found in the total number of hCD8⁺ T cells (FIG. 2C). To compare the functional capacity fetal liver or cord blood-derived human HPCs in mounting adaptive humoral response, the capacity of humanized NSG-SGM3F-A2 mice to produce human antibodies was assessed. To this end, total human Ig in the plasma at 12-week post-transplant by ELISA was measured. As shown in FIG. 2D, both groups of mice secreted comparable amount of human IgM in the plasma and the level of total human IgG and IgA subclass were also similar. Thus, the capacity of antibody secretion and Ig class switch were comparable between difference sources of HPCs in humanized NSG-SGM3F-A2 mice. Overall, a higher level of variability in mice generated among different donors from the same source of HPCs than the variability between the sources of HPCs was observed. This analysis showed that cord blood HPCs provided comparable human engraftment to fetal liver HPCs in NSG-SGM3F-A2 mice.

Generation of Mouse Model SGM3F: NSG-SGM3-Flt3ko or SGM3F mice (NOD.Cg-Prkdc^{scid} Il2rg^{tm1Wjl}-Flt3^{em1AkP} Tg(CMV-IL3,CSF2,KITLG)^{1Eav/MloySzJ}), were generated by crossing NSG-SGM3 mice (NOD.Cg-Prkdc^{scid}Il2rg^{tm1Wjl} Tg(CMV-IL3,CSF2,KITLG)^{1Eav/MloySzJ}. RRID:IMSR JAX:013062) to NSGF (NOD.Cg-Prkdcscid Il2rg^{tm1Wjl}-Flt3^{em1Akp}) mice and interbred until all offspring were homozygous. NSG-SGM3 mice carried three separate transgenes which were designed each carrying either the human interleukin-3 (IL3) gene, the human granulocyte/macrophage-stimulating factor (GM-CSF) gene, or human stem cell factor (SCF) gene. Expression of each gene is driven by a human cytomegalovirus promoter/enhancer sequence and is followed by a human growth hormone cassette and a polyadenylation (polyA) sequence. The transgenes were microinjected into fertilized C57BL/6xC3H/HeN oocytes. The resulting founders, carrying all three transgenes (3GS) were backcrossed to BALB/c-scid/scid mice for several generations and subsequently backcrossed to NOD.CB17-Prkdcscid mice for at least 11 generations (Nicolini et al., 2004). These mice were bred to NSG mice (NOD.Cg-Prkdc^{scid} Il2rg^{tm1Wjl}; RRID:IMSR JAX: 005557), and were then interbred until all offspring were homozygous for 3GS and the IL2rg targeted mutation. Upon arrival at The Jackson Laboratory, transgenic mice were bred to NSG mice for one generation to establish NSG-SGM3 mice. NSGF mice were generated using CRISPR/cas system. were generated by CRISPR using Cas9 mRNA and sgRNAs (5′-AAGTGCAGCTCGCCACCCCA-3′, SEQ ID NO: 2) targeting exon 3 of mouse Flt3 in fertilized eggs of NSG mice. The blastocysts derived from the injected embryos were transplanted into foster mothers and newborn pups were obtained. Mice carrying a null deletion were backcrossed to NSG. F0 and F1 littermates were tail tipping and tested for successful gene-knockout by PCR and Sanger sequencing. Primers (5′-GGTACCAGCAGAGTTGGATAGC-3′, SEQ ID NO: 3) and (5′-ATCCCTTACACAGAAGCTGGAG-3′, SEQ ID NO: 4) were used in a PCR reaction to detect the mouse Flt3 wildtype allele from mutant allele (Table 1). The WT allele yields a DNA fragment 799 bp in length, whereas the mutated allele generates a DNA fragment of 363 bp in length.

Generation of Mouse Model NSG-SGM3F-A2: NSG-SGM3-Flt3ko-A2 or NSG-SGM3F-A2 mice (NOD.Cg-Prkdc^{scid}Il2rg^{tm1Wjl} Tg(HLA-A/H2-D/B2M)^{1Dvs/SzJ} Flt3^{em1Akp} Tg(CMV-IL3,CSF2,KITLG)^{1Eav/MloySzJ}) were generated by crossing NSG-HLA-A2/HHD mice (RRID:IMSR JAX: 014570) to NSG-SGM3-Flt3ko or SGM3F mice (NOD.Cg-Prkdc^{scid} Il2rg^{tm1Wjl}-Flt3^{em1AkP} Tg(CMV-IL3,CSF2,KITLG)^{1Eav/MloySzJ}) NSG-SGM3-Flt3ko mice carried three separate transgenes which were designed each carrying either the human interleukin-3 (IL3) gene, the human granulocyte/macrophage-stimulating factor (GM-CSF) gene, or human stem cell factor (SCF) gene. Expression of each gene is driven by a human cytomegalovirus promoter/enhancer sequence and is followed by a human growth hormone cassette and a polyadenylation (polyA) sequence. The transgenes were microinjected into fertilized C57BL/6xC3H/HeN oocytes. The resulting founders, carrying all three transgenes (3GS) were backcrossed to BALB/c-scid/scid mice for several generations and subsequently backcrossed to NOD.CB17-Prkdcscid mice for at least 11 generations (Nicolini et al., 2004). These mice were bred to NSG mice (NOD.Cg-Prkdcscid Il2rg^{tm1Wjl}; RRID:IMSR JAX: 005557) and were then interbred until all offspring were homozygous for 3GS and the IL2rg targeted mutation. Upon arrival at The Jackson Laboratory, transgenic mice were bred to NSG mice for one generation to establish NSG-SGM3 mice. NSGF mice were generated using CRISPR/cas system. Cas9 mRNA and sgRNAs targeting mouse Flt3 were coinjected into fertilized NSG oocytes. The resulting founders, carrying Flt3 deletion were bred to NSG mice, and were then interbred until all offspring were homozygous for Flt3 targeted mutation. The NSG-SGM3 mice were bred to NSGF mice for 2 generation to establish NSG-SGM3-Flt3ko mice. NSG-HLA-A2/HHD mice carried HLA-A2/H2-D/B2M transgene encoded a human B2-microglubulin (B2M) covalently linked to the MHC class 1, alpha1 and alpha2 binding domains of the human HLA-A2.1 gene, and the alpha3, cytoplasmic and transmembrane domains of the murine H2-db (Pascolo et al., 1997; Shultz et al., 2010). The NSG-HLA-A2/HHD mice were bred to NSG-SGM3F mice for 4 generation to establish NSG-SGM3F-A2 mice.

### Additional Material and Methods

### Humanized Mice

Humanized mice were generated on different strains of mice in NSG background obtained from The Jackson Laboratory (Bar Harbor, Me.). All protocols were reviewed and approved by the Institutional Animal Care and Use Committee at The Jackson Laboratory (14005) and University of Connecticut Health Center (101163-0220 & 101831-0321; Farmington, Conn.). Mice were sub-lethally irradiated (10 cGy per gram of body weight) using gamma irradiation at the age of four weeks. 100,000 CD34⁺ HPCs from fetal liver or full-term cord blood (Advanced Bioscience Resources or Lonza) were given by tail-vein intravenous (IV) injection in 200 μL of PBS. Alternatively, mice received adult CD34⁺ HPCs from bone marrow (Lonza) as indicated. Mice were bled at 4-12 weeks post HPC transplant to evaluate engraftment and euthanized according to the individual experimental design.

### Flow Cytometry Analysis

Mice were euthanized and blood was collected with heparin. The bone (femur and tibia), spleen and lungs were collected to make single cell suspension. Spleen were digested with 50 μg/ml of Liberase (Roche Diagnostics, Indianapolis, Ind.) and 24 U/mL of DNase I (Sigma) for 10 min at 37° C. Lungs were digested with 50 μg/ml of Liberase and 24 U/mL of DNase I (Sigma) for 30 min at 37° C., followed by mechanical dissociation with GentleMACS (Miltenyi Biotec). Cells were first treated with murine Fc blocker (BD) and then stained on ice with antibody cocktails for 30 mins. After washing twice with PBS, the samples were acquired on a LSRII or FACSARIA II (BD), and analyzed with FlowJo software (Tree Star, Ashland, Oreg.). For the expression of human HLA-0201, cells were stained with antibodies to mouse CD45-BV421 (30-F11, BD) and human HLA-A2-PE (BB7.2, BD). For human engraftment in the blood, cells were stained with antibodies to mouse CD45-BV650 (30-F11, BD) and human CD45-BV510 (HI30, BD), CD33-PE (P67.6, Biolegend), CD14-PE-Cy7 (MqP9, BD), CD19-APC (HIB19, Biolegend) and CD3-APC-H7 (SK7, BD).

### Immunofluorescence Staining

Tissues were embedded in OCT (Sakura Finetek U.S.A.) and snap frozen in liquid nitrogen. Frozen sections were cut at 6 μm, air dried on Superfrost plus slides and fixed with cold acetone for five minutes. Tissue sections were first treated with 0.03% hyaluronidase (Sigma) for 15 minutes, followed by treatment with Background Buster and Fc Receptor Block (Innovex Bioscience). The sections were then stained with monoclonal antibodies to human CD3 (UCHT1, Biolegend), CD11c (S-HCL-3, BD), HLA-A2 (BB7.2, BD), HLA-DR (L243, Biolegend) or pan-cytokeratin (AE1/AE3, Miltenyi Biotech) for one hour at room temperature, followed by isotype-specific secondary antibodies for 30 minutes at room temperature. Respective isotype antibodies were used as the control. Finally, sections were counterstained with 1 μg/ml of 4′,6-diamidino-2-phenylindole (DAPI), mounted with Fluoromount (Thermo Fisher Scientific), and visualized using a Leica SP 8 confocal microscope with Leica LAS AF 2.0 software or a Zeiss Axio fluorescence microscope with ZEN software.

### Statistical Analysis

Statistical analysis will be performed in Prism (GraphPad). Comparisons between any 2 groups are analyzed using the Mann-Whitney test or two-tailed t-test. Comparisons between any 3 or more groups are analyzed by analysis of variance (ANOVA).

TABLE 1
List of primers for mouse genotype.

| Allele | Name | Sequence |
|---|---|---|
| Flt3 | mFlt3_F | GGTACCAGCAGAGTTGGATAGC |
| | | (SEQ ID NO: 3) |
| Flt3 | mFlt3_R | ATCCCTTACACAGAAGCTGGAG |
| | | (SEQ ID NO: 4) |

SEQUENCES

SEQ ID NO: 1, Flt3^{em1Akp}
GGGCACGTGGGATCGGCTGCAGCACTGCGCCAGTTCAGCCCGCCTAGCAGCGAGCG
GCCGCGGCCTCTGGAGAGAGGTTCCTCCCCCTCTGCTCTGCACCAGTCCGAGGGAAT
CTGTGGTCAGTGACGCGCATCCTTCAGCGAGCCACCTGCAGCCCGGGGCGCGCCGC
TGGGACCGCATCACAGGCTGGGCCGGCGGCCTGGCTACCGCGCGCTCCGGAGGCCA
TGCGGGCGTTGGCGCAGCGCAGCGACCGGCGGCTGCTGCTGCTTGTTGTTTTGTCAG
TAATGATTCTTGAGACCGTTACAAACCAAGACCTGCCTGTGATCAAGTGTGTTTTAA
TCAGTCATGAGAACAATGGCTCATCAGCGGGAAAGCCATCATCGTACCGAATGAGG
AATCGTTTCCATGGCCATCTTGAACGTGACAGAGACCCAGGCAGGAGAATACCTAC
TCCATATTCAGAGCGAAGCCGCCAACTACACAGTACTGTTCACAGTGAATGTAAGA
GATACACAGCTGTACGTGCTAAGAAGACCTTACTTTAGGAAGATGGAAAACCAGGA
CGCACTGCTCTGCATCTCCGAGGGTGTTCCAGAGCCCACTGTGGAGTGGGTGCTCTG
CAGCTCCCACAGGGAAAGCTGTAAAGAAGAAGGCCCTGCTGTTGTCAGAAAGGAG
GAAAAGGTACTTCATGAGTTGTTCGGAACAGACATCAGATGCTGTGCTAGAAATGC
ACTGGGCCGCGAATGCACCAAGCTGTTCACCATAGATCTAAACCAGGCTCCTCAGA
GCACACTGCCCCAGTTATTCCTGAAAGTGGGGGAACCCTTGTGGATCAGGTGTAAG
GCCATCCATGTGAACCATGGATTCGGGCTCACCTGGGAGCTGGAAGACAAAGCCCT
GGAGGAGGGCAGCTACTTTGAGATGAGTACCTACTCCACAAACAGGACCATGATTC
GGATTCTCTTGGCCTTTGTGTCTTCCGTGGGAAGGAACGACACCGGATATTACACCT
GCTCTTCCTCAAAGCACCCCAGCCAGTCAGCGTTGGTGACCATCCTAGAAAAAGGG
TTTATAAACGCTACCAGCTCGCAAGAAGAGTATGAAATTGACCCGTACGAAAAGTT
CTGCTTCTCAGTCAGGTTTAAAGCGTACCCACGAATCCGATGCACGTGGATCTTCTC
TCAAGCCTCATTTCCTTGTGAACAGAGAGGCCTGGAGGATGGGTACAGCATATCTA
AATTTTGCGATCATAAGAACAAGCCAGGAGAGTACATATTCTATGCAGAAAATGAT
GACGCCCAGTTCACCAAAATGTTCACGCTGAATATAAGAAAGAAACCTCAAGTGCT
AGCAAATGCCTCAGCCAGCCAGGCGTCCTGTTCCTCTGATGGCTACCCGCTACCCTC
TTGGACCTGGAAGAAGTGTTCGGACAAATCTCCCAATTGCACGGAGGAAATCCCAG
AAGGAGTTTGGAATAAAAAGGCTAACAGAAAAGTGTTTGGCCAGTGGGTGTCGAGC
AGTACTCTAAATATGAGTGAGGCCGGGAAAGGGCTTCTGGTCAAATGCTGTGCGTA
CAATTCTATGGGCACGTCTTGCGAAACCATCTTTTTAAACTCACCAGGCCCCTTCCC
TTTCATCCAAGACAACATCTCCTTCTATGCGACCATTGGGCTCTGTCTCCCCTTCATT
GTTGTTCTCATTGTGTTGATCTGCCACAAATACAAAAAGCAATTTAGGTACGAGAGT
CAGCTGCAGATGATCCAGGTGACTGGCCCCCTGGATAACGAGTACTICTACGTTGAC
TTCAGGGACTATGAATATGACCTTAAGTGGGAGTTCCCGAGAGAGAACTTAGAGTT
TGGGAAGGTCCTGGGGTCTGGCGCTTTCGGGAGGGTGATGAACGCCACGGCCTATG
GCATTAGTAAAACGGGAGTCTCAATTCAGGTGGCGGTGAAGATGCTAAAAGAGAAA
GCTGACAGCTGTGAAAAAGAAGCTCTCATGTCGGAGCTCAAAATGATGACCCACCT
GGGACACCATGACAACATCGTGAATCTGCTGGGGGCATGCACACTGTCAGGGCCAG
TGTACTTGATTTTTGAATATTGTTGCTATGGTGACCTCCTCAACTACCTAAGAAGTA
AAAGAGAGAAGTTTCACAGGACATGGACAGAGATTTTTAAGGAACATAATTTCAGT
TTTTACCCTACTTTCCAGGCACATTCAAATTCCAGCTTCAGAATGAATTAAATTCCC
ATTGAACCCTGAGAGCTGATCCAAGGGCGGGTGTAACTGAACTTCTCGTGAACCAG
GCATGATGAGATTGAATATGAAAACCAGAAGAGGCTGGCAGAAGAAGAGGAGGAA
GATTTGAACGTGCTGACGTTTGAAGACCTCCTTTGCTTTGCGTACCAAGTGGCCAAA
GGCATGGAATTCCTGGAGTTCAAGTCGTGTGTCCACAGAGACCTGGCAGCCAGGAA
TGTGTTGGTCACCCACGGGAAGGTGGTGAAGATCTGTGACTTTGGACTGGCCCGAG
ACATCCTGAGCGACTCCAGCTACGTCGTCAGGGGCAACGCACGGCTGCCGGTGAAG
TGGATGGCACCTGAGAGCTTATTTGAAGGGATCTACACAATCAAGAGTGACGTCTG
GTCCTACGGCATCCTTCTCTGGGAGATATTTTCACTGGGTGTGAACCCTTACCCTGG
CATTCCTGTCGACGCTAACTTCTATAAACTGATTCAGAGTGGATTTAAAATGGAGCA
GCCATTCTATGCCACAGAAGGGATATGTATCAGAACATGGGTGGCAACGTCCCAGA
ACATCCATCCATCTACCAAAACAGGCGGCCCCTCAGCAGAGAGGCAGGCTCAGAGC
CGCCATCGCCACAGGCCCAGGTGAAGATTCACGGAGAAAGAAGTTAGCGAGGAGG
CCTTGGACCCCGCCACCCTAGCAGGCTGTAGACCACAGAGCCAAGATTAGCCTCGC
CTCTGAGGAAGCGCCCTACAGGCCGTTGCTTCGCTGGACTTTTCTCTAGATGCTGTC
TGCCATTACTCCAAAGTGACTTCTATAAAATCAAACCTCTCCTCGCACAGGTGGGAG
AGCCAATAATGAGACTTGTTGGTGAGCCCGCCTACCCTGGGGGGCCTTTCCAGGCCC
CCCAGGCTTGAGGGGAAAGCCATGTATCTGAAATATAGTATATTCTTGTAAATACGT
GAAACAAACCAAACCCGTTTTTTGCTAAGGGAAAGCTAAATATGATTTTTAAAAAT
CTATGTTTTAAAATACTATGTAACTTTTTCATCTATTTAGTGATATATTTTATGGATG
GAAATAAACTTTCTACTGTAGAAA
SEQ ID NO: 2, gRNA for mouse Flt3, 5′-AAGTGCAGCTCGCCACCCCA-3′
SEQ ID NO: 3-4, PCR primers for mouse Flt3 including
5′-GGTACCAGCAGAGTTGGATAGC-3′ (SEQ ID NO: 3) and
5′-ATCCCTTACACAGAAGCTGGAG-3′ (SEQ ID NO: 4)

### REFERENCES

Aspord, C., Pedroza-Gonzalez, A., Gallegos, M., Tindle, S., Burton, E. C., Su, D., Palucka, A. K. (2007). Breast cancer instructs dendritic cells to prime interleukin 13-secreting CD4+ T cells that facilitate tumor development. J Exp Med, 204(5), 1037-1047. doi:10.1084/jem.20061120
Billerbeck, E., Horwitz, J. A., Labitt, R. N., Donovan, B. M., Vega, K., Budell, W. C., Ploss, A. (2013). Characterization of human antiviral adaptive immune responses during hepatotropic virus infection in HLA-transgenic human immune system mice. J Immunol, 191(4), 1753-1764. doi:10.4049/jimmunol.1201518
Blunt, T., Finnie, N. J., Taccioli, G. E., Smith, G. C., Demengeot, J., Gottlieb, T. M., Jackson, S. P. (1995). Defective DNA-dependent protein kinase activity is linked to V(D)J recombination and DNA repair defects associated with the murine scid mutation. Cell, 80(5), 813-823. doi:10.1016/0092-8674(95)90360-7
Cao, X., Shores, E. W., Hu-Li, J., Anver, M. R., Kelsall, B. L., Russell, S. M., et al. (1995). Defective lymphoid development in mice lacking expression of the common cytokine receptor gamma chain. Immunity, 2(3), 223-238. doi:10.1016/1074-7613(95)90047-0
Carroll, D. (2011). Genome engineering with zinc-finger nucleases. Genetics, 188(4), 773-782. doi:10.1534/genetics.111.131433
Danner, R., Chaudhari, S. N., Rosenberger, J., Surls, J., Richie, T. L., Brumeanu, T. D., & Casares, 5. (2011). Expression of HLA class II molecules in humanized NOD.Rag1KO.IL2RgcKO mice is critical for development and function of human T and B cells. PLoS One, 6(5), e19826. doi:10.1371/journal.pone.0019826
Deltcheva, E., Chylinski, K., Sharma, C. M., Gonzales, K., Chao, Y., Pirzada, Z. A., Charpentier, E. (2011). CRISPR RNA maturation by trans-encoded small RNA and host factor RNase III. Nature, 471(7340), 602-607. doi:10.1038/nature09886
Gaj, T., Gersbach, C. A., & Barbas, C. F., 3rd. (2013). ZFN, TALEN, and CRISPR/Cas-based methods for genome engineering. Trends Biotechnol, 31(7), 397-405. doi:10.1016/j.tibtech.2013.04.004
Gordon, J. W., & Ruddle, F. H. (1981). Integration and stable germ line transmission of genes injected into mouse pronuclei. Science, 214(4526), 1244-1246. doi:10.1126/science.6272397
Gossler, A., Doetschman, T., Korn, R., Seffling, E., & Kemler, R. (1986). Transgenesis by means of blastocyst-derived embryonic stem cell lines. Proc Natl Acad Sci USA, 83(23), 9065-9069. doi:10.1073/pnas.83.23.9065
Greiner, D. L., Hesselton, R. A., & Shultz, L. D. (1998). SCID mouse models of human stem cell engraftment. Stem Cells, 16(3), 166-177. doi:10.1002/stem.160166
Hagai, T., Chen, X., Miragaia, R. J., Rostom, R., Gomes, T., Kunowska, N., Teichmann, S. A. (2018). Gene expression variability across cells and species shapes innate immunity. Nature, 563(7730), 197-202. doi:10.1038/s41586-018-0657-2
Harms, D. W., Quadros, R. M., Seruggia, D., Ohtsuka, M., Takahashi, G., Montoliu, L., & Gurumurthy, C. B. (2014). Mouse Genome Editing Using the CRISPR/Cas System. Curr Protoc Hum Genet, 83, 15 17 11-27. doi:10.1002/0471142905.hg1507s83
Inui, M., Miyado, M., Igarashi, M., Tamano, M., Kubo, A., Yamashita, S., Takada, S. (2014). Rapid generation of mouse models with defined point mutations by the CRISPR/Cas9 system. Sci Rep, 4, 5396. doi:10.1038/srep05396
Jaenisch, R. (1976). Germ line integration and Mendelian transmission of the exogenous Moloney leukemia virus. Proc Natl Acad Sci USA, 73(4), 1260-1264. doi:10.1073/pnas.73.4.1260
Jinek, M., Chylinski, K., Fonfara, I., Hauer, M., Doudna, J. A., & Charpentier, E. (2012). A programmable dual-RNA-guided DNA endonuclease in adaptive bacterial immunity. Science, 337(6096), 816-821. doi:10.1126/science.1225829
Joung, J. K., & Sander, J. D. (2013). TALENs: a widely applicable technology for targeted genome editing. Nat Rev Mol Cell Biol, 14(1), 49-55. doi:10.1038/nrm3486
Kanazawa, N. (2007). Dendritic cell immunoreceptors: C-type lectin receptors for pattern-recognition and signaling on antigen-presenting cells. J Dermatol Sci, 45(2), 77-86.
Makino, S., Kunimoto, K., Muraoka, Y., Mizushima, Y., Katagiri, K., & Tochino, Y. (1980). Breeding of a non-obese, diabetic strain of mice. Jikken Dobutsu, 29(1), 1-13. doi:10.1538/expanim1978.29.1_1
Matsumura, T., Kametani, Y., Ando, K., Hirano, Y., Katano, I., Ito, R., Habu, S. (2003). Functional CD5+ B cells develop predominantly in the spleen of NOD/SCID/gammac(null) (NOG) mice transplanted either with human umbilical cord blood, bone marrow, or mobilized peripheral blood CD34+ cells. Exp Hematol, 31(9), 789-797.
Mestas, J., & Hughes, C. C. (2004). Of mice and not men: differences between mouse and human immunology. J Immunol, 172(5), 2731-2738.
Najima, Y., Tomizawa-Murasawa, M., Saito, Y., Watanabe, T., Ono, R., Ochi, T., Ishikawa, F. (2016). Induction of WT1-specific human CD8+ T cells from human HSCs in HLA class I Tg NOD/SCID/IL2rgKO mice. Blood, 127(6), 722-734. doi:10.1182/blood-2014-10-604777
Nicolini, F. E., Cashman, J. D., Hogge, D. E., Humphries, R. K., & Eaves, C. J. (2004). NOD/SCID mice engineered to express human IL-3, GM-CSF and Steel factor constitutively mobilize engrafted human progenitors and compromise human stem cell regeneration. Leukemia, 18(2), 341-347. doi:10.1038/sj.leu.2403222
Pascolo, S., Bervas, N., Ure, J. M., Smith, A. G., Lemonnier, F. A., & Perarnau, B. (1997). HLA-A2.1-restricted education and cytolytic activity of CD8(+) T lymphocytes from beta2 microglobulin (beta2m) HLA-A2.1 monochain transgenic H-2Db beta2m double knockout mice. J Exp Med, 185(12), 2043-2051. doi:10.1084/jem.185.12.2043
Pedroza-Gonzalez, A., Xu, K., Wu, T. C., Aspord, C., Tindle, S., Marches, F., Palucka, A. K. (2011). Thymic stromal lymphopoietin fosters human breast tumor growth by promoting type 2 inflammation. J Exp Med, 208(3), 479-490. doi:10.1084/jem.20102131
Shultz, L. D., Brehm, M. A., Garcia-Martinez, J. V., & Greiner, D. L. (2012). Humanized mice for immune system investigation: progress, promise and challenges. Nat Rev Immunol, 12(11), 786-798. doi:10.1038/nri3311
Shultz, L. D., Ishikawa, F., & Greiner, D. L. (2007). Humanized mice in translational biomedical research. Nat Rev Immunol, 7(2), 118-130. doi:10.1038/nri2017
Shultz, L. D., Lyons, B. L., Burzenski, L. M., Gott, B., Chen, X., Chaleff, S., Handgretinger, R. (2005). Human lymphoid and myeloid cell development in NOD/LtSz-scid IL2R gamma null mice engrafted with mobilized human hemopoietic stem cells. J Immunol, 174(10), 6477-6489. doi:10.4049/jimmunol.174.10.6477
Shultz, L. D., Saito, Y., Najima, Y., Tanaka, S., Ochi, T., Tomizawa, M., Ishikawa, F. (2010). Generation of functional human T-cell subsets with HLA-restricted immune responses in HLA class I expressing NOD/SCID/IL2r gamma(null) humanized mice. Proc Natl Acad Sci USA, 107(29), 13022-13027. doi:10.1073/pnas.1000475107
Shultz, L. D., Schweitzer, P. A., Christianson, S. W., Gott, B., Schweitzer, I. B., Tennent, B., et al. (1995). Multiple defects in innate and adaptive immunologic function in NOD/LtSz-scid mice. J Immunol, 154(1), 180-191.
Takaki, T., Marron, M. P., Mathews, C. E., Guttmann, S. T., Bottino, R., Trucco, M., Serreze, D. V. (2006). HLA-A*0201-restricted T cells from humanized NOD mice recognize autoantigens of potential clinical relevance to type 1 diabetes. J Immunol, 176(5), 3257-3265. doi:10.4049/jimmunol.176.5.3257
Theocharides, A. P., Rongvaux, A., Fritsch, K., Flavell, R. A., & Manz, M. G. (2016). Humanized hemato-lymphoid system mice. Haematologica, 101(1), 5-19. doi:10.3324/haematol.2014.115212
Traggiai, E., Chicha, L., Mazzucchelli, L., Bronz, L., Piffaretti, J. C., Lanzavecchia, A., & Manz, M. G. (2004). Development of a human adaptive immune system in cord blood cell-transplanted mice. Science, 304(5667), 104-107. doi:10.1126/science.1093933 Victor Garcia, J. (2016). Humanized mice for HIV and AIDS research. Curr Opin Virol, 19, 56-64. doi:10.1016/j.coviro.2016.06.010
Williams, A., Flavell, R. A., & Eisenbarth, S. C. (2010). The role of NOD-like Receptors in shaping adaptive immunity. Curr Opin Immunol, 22(1), 34-40. doi:10.1016/j.coi.2010.01.004
Wu, T. C., Xu, K., Banchereau, R., Marches, F., Yu, C. I., Martinek, J., Palucka, K. (2014). Reprogramming tumor-infiltrating dendritic cells for CD103+ CD8+ mucosal T-cell differentiation and breast cancer rejection. Cancer Immunol Res, 2(5), 487-500. doi:10.1158/2326-6066.CIR-13-0217
Wu, T. C., Xu, K., Martinek, J., Young, R. R., Banchereau, R., George, J., Palucka, A. K. (2018). IL1 Receptor Antagonist Controls Transcriptional Signature of Inflammation in Patients with Metastatic Breast Cancer. Cancer Res, 78(18), 5243-5258. doi:10.1158/0008-5472.CAN-18-0413
Wunderlich, M., Chou, F. S., Link, K. A., Mizukawa, B., Perry, R. L., Carroll, M., & Mulloy, J. C. (2010). AML xenograft efficiency is significantly improved in NOD/SCID-IL2RG mice constitutively expressing human SCF, GM-CSF and IL-3. Leukemia, 24(10), 1785-1788. doi:10.1038/leu.2010.158
Yu, C. I., Gallegos, M., Marches, F., Zurawski, G., Ramilo, O., Garcia-Sastre, A., Palucka, A. K. (2008). Broad influenza-specific CD8+ T-cell responses in humanized mice vaccinated with influenza virus vaccines. Blood, 112(9), 3671-3678. doi:10.1182/blood-2008-05-157016
Zhang, L., & Su, L. (2012). HIV-1 immunopathogenesis in humanized mouse models. Cell Mol Immunol, 9(3), 237-244. doi:10.1038/cmi.2012.7

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

What is claimed is:

1. A non-obese diabetic (NOD) mouse comprising
an inactivated mouse Prkdc allele;
an inactivated mouse IL2rg allele;
an inactivated mouse Flt3 allele;
a nucleic acid encoding human interleukin 3 (IL3);
a nucleic acid encoding human granulocyte/macrophage-stimulating factor (GM-CSF);
a nucleic acid encoding human stem cell factor (SCF); and
a nucleic acid encoding a human B2-microglubulin (B2M) covalently linked to MHC class 1, alpha1, and alpha2 binding domains of a human HLA-A2.1 gene, and alpha3 cytoplasmic and transmembrane domains of murine H2-db (HLA-A2/H2-D/B2M).

2. The mouse of claim 1, wherein the mouse is a NOD.Cg-Prkdc^{scid} Il2rg^{tm1Wjl}/SzJ (NOD scid gamma) mouse comprising an inactivated mouse Flt3 allele; a nucleic acid encoding human IL3; a nucleic acid encoding human GM-CSF; a nucleic acid encoding human SCF; and a nucleic acid encoding HLA-A2/H2-D/B2M.

3. The mouse of claim 1 or 2, wherein the nucleic acid encoding HLA-A2/H2-D/B2M comprises a transgene encoding HLA-A2/H2-D/B2M.

4. The mouse of any one of claims 1-3, wherein the mouse expresses the transgene encoding HLA-A2/H2-D/B2M.

5. The mouse of any one of claims 1-4, wherein bone marrow mouse CD45⁺ cells express a detectable level of HLA-A2 at 4 weeks of age.

6. The mouse of any one of claims 1-5, wherein the mouse has been irradiated, transplanted with human hematopoietic progenitor cells (HPCs), and the human HPCs engraft as human CD45⁺ cells.

7. The mouse of claim 6, wherein the human HPCs are from fetal liver, cord blood, or bone marrow, and the engrafted human CD45⁺ cells in the mouse comprise a mixed population of CD19⁺ B cells, CD33⁺ myeloid cells, and CD3⁺ T cells.

8. The mouse of claim 6, wherein the human HPCs are from fetal liver, cord blood, or bone marrow, and lung tissue of the mouse comprises CD3⁺ T cells and HLA-DR⁺ CD11c⁺ dendritic cells.

9. The mouse of claim 6, wherein the human HPCs are HLA-A2⁺ and the mouse comprises HLA-A2⁺ mouse thymic epithelial cells.

10. A method of producing the mouse of any one of claims 1-5 comprising introducing the transgene encoding HLA-A2/H2-D/B2M into a NOD scid gamma mouse that comprises an inactivated mouse Flt3 allele, a nucleic acid encoding human interleukin 3 (IL3), a nucleic acid encoding human granulocyte/macrophage-stimulating factor (GM-CSF), and a nucleic acid encoding human stem cell factor (SCF).

11. A method of producing the mouse of any one of claims 1-5 comprising crossing a NSG-SGM3F mouse that comprises a nucleic acid encoding human interleukin 3 (IL3), a nucleic acid encoding human granulocyte/macrophage-stimulating factor (GM-CSF), a nucleic acid encoding human stem cell factor (SCF), and an inactivated mouse Flt3 allele with an NSG-HLA-A2/HHD mouse comprising a transgene encoding a human B2-microglubulin (B2M) covalently linked to the MHC class 1, alpha1 and alpha2 binding domains of the human HLA-A2.1 gene, and the alpha3, cytoplasmic and transmembrane domains of the murine H2-db.

12. A method of producing the mouse of any one of claims 1-5, comprising:
(a) developing founder mice that have a NOD scid gamma genetic background, an inactivated mouse Flt3 allele, a nucleic acid encoding human IL3, a nucleic acid encoding human GM-CSF, and a nucleic acid encoding human SCF;
(b) breeding the founder mice to NOD scid gamma mice that comprise a transgene encoding a human B2-microglubulin (B2M) covalently linked to the MHC class 1, alpha1 and alpha2 binding domains of the human HLA-A2.1 gene, and the alpha3, cytoplasmic and transmembrane domains of the murine H2-db to produce F1 progeny mice; and
(c) interbreeding the F1 progeny mice to produce F2 progeny mice homozygous for the inactivated Flt3 allele, a nucleic acid encoding human interleukin 3 (IL3), a nucleic acid encoding human granulocyte/macrophage-stimulating factor (GM-CSF), a nucleic acid encoding human stem cell factor (SCF), and a transgene encoding a human B2-microglubulin (B2M) covalently linked to the MHC class 1, alpha1 and alpha2 binding domains of the human HLA-A2.1 gene, and the alpha3, cytoplasmic and transmembrane domains of the murine H2-db.

13. A method comprising breeding female mice homozygous for Prkdc^{scid}, homozygous for Il2rg^{tm1Wjl}, homozygous for Flt3^{em1Akp}, homozygous for Il-3, homozygous for GM-CSF, homozygous for SCF, and homozygous for a transgene encoding HLA-A2/H2-D/B2M with male mice homozygous for Prkdc^{scid}, hemizygous for the X-linked Il2rg^{tm1Wjl}, homozygous for Flt3^{em1Akp}, homozygous for Il-3, homozygous for GM-CSF, homozygous for SCF, and homozygous for a transgene encoding HLA-A2/H2-D/B2M and to produce progeny mice.

14. A cell obtained from the mouse of any one of the preceding claims.

15. A mouse comprising a cell having the same genotype of a cell obtained from the mouse of any one of the preceding claims.

16. A progeny mouse of the mouse of any one of the preceding claims.

17. A method of producing the mouse of any one of the preceding claims.

18. A method of propagating the mouse of any one of the preceding claims.

19. The method of claim 18 comprising breeding the mouse of any one of the preceding claims with a second mouse to produce a progeny mouse.

20. The method of claim 19, wherein the second mouse is a mouse of any one of the preceding claims.

21. A method comprising sublethally irradiating the mouse of any one of the preceding claims to produce an irradiated mouse.

22. The method of claim 21 further comprising administering to the mouse human hematopoietic progenitor cells (HPCs).

23. The method of claim 21 or 22 further comprising administering to the mouse an agent of interest.

24. The method of claim 23 further comprising assessing an effect of the agent on human immune cells in the mouse.

25. The method of claim 24, wherein the human immune cells are selected from T cells, dendritic cells, natural killer cells, and macrophages.
